(19) United States
(12) Patent Application Publication
(10) Pub. No.: US 2026/0115358 A1
(43) Pub. Date: Apr. 30, 2026

(54) LOW-SWELLING DECELLULARIZED CORNEA, PREPARATION METHOD THEREFOR AND USE

(71) Applicant: Eye Institute of Shandong First Medical University, Shandong (CN)

(72) Inventors: Weiyun SHI, Shandong (CN); Long ZHAO, Shandong (CN); Ting WANG, Shandong (CN); Qingjun ZHOU, Shandong (CN); Zhen SHI, Shandong (CN)

(21) Appl. No.: 19/149,002

(22) PCT Filed: Aug. 12, 2024
(86) PCT No.: PCT/CN2024/111354

§ 371 Date: Jul. 17, 2025

(30) Foreign Application Priority Data
Aug. 28, 2023 (CN) 202311090123.4
Aug. 28, 2023 (CN) 202311090200.6

(51) Int. Cl. A61L 27/36

(52) U.S. Cl. CPC A61L 27/3604; A61L 27/3695; A61L 2430/16

(57) ABSTRACT

The present invention discloses a low-swelling decellularized cornea, its preparation method, and applications. The decellularized cornea is obtained by incorporating methacrylated compounds into a decellularized corneal matrix. The decellularized cornea exhibits thermosensitive and photopolymerizable properties, capable of exuding monomer polymers at temperatures above 25° C. and forming a stable biological adhesion layer on the recipient bed through photopolymerization. The decellularized cornea features abundant material sources and enables rapid trauma repair without surgical sutures.

### TECHNICAL FIELD

The present invention belongs to the field of tissue engineering and regenerative medicine, and particularly relates to a low-swelling decellularized cornea, and a preparation method and application thereof.

### BACKGROUND

Keratoplasty is a last resort for treating patients with corneal blindness. The surgical methods of corneal transplantation include penetrating keratoplasty, lamellar keratoplasty and deep lamellar keratoplasty. Penetrating keratoplasty, referring to full-layer corneal transplantation including corneal endothelium, replaces diseased and defective cornea with intact and healthy cornea, which not only restores the integrity of the eyeball and controls keratopathy, but also greatly improves visual acuity. The surgical indications of penetrating keratoplasty are widely divided into infectious keratitis, ocular trauma, bullous keratopathy, secondary keratoplasty, keratoconus, corneal tumors, corneal degeneration or dystrophy, nibbling corneal ulcer and other corneal diseases. At present, penetrating keratoplasty is the most common surgical method for corneal transplantation in China. Although this surgery has a high success rate, the quality requirements for donor corneas that are intended for penetrating keratoplasty are exceptionally rigorous, in which healthy endothelium is a must to prevent corneal edema and loss of clarity after transplantation. Due to the influence of ethical, moral and social customs in China, there is a critical shortage of the donor corneas, and most of the corneal blinded patients cannot be timely treated.

Although artificial corneas can be used for penetrating keratoplasty, the artificial corneas are mainly used for surgical rehabilitation of patients with end-stage ocular surface diseases, such as severe ocular chemical injury and thermal burn, limbal stem cell deficiency, extensive corneal vascularization and autoimmune diseases. The artificial corneas are generally made of high molecular synthetic materials, which cannot bio-integrate with the recipient tissue to achieve tissue regeneration, and are prone to postoperative complications such as retinal detachment, corneal calcification, glaucoma, corneal lysis, prosthesis prolapse, aqueous humor leakage and severe cataract.

In recent years, the availability of decellularized cornea products has alleviated the shortage of the donor corneas. However, due to the hydrophilic swelling tendency of fresh corneal stroma, the decellularized cornea cannot directly contact the aqueous humor. Thus, its clinical application is limited to repairing corneal ulcers not involving the full thickness.

Furthermore, sutureless implantation of the corneal donor graft is clinically preferable, as it significantly reduces patient discomfort, infection risk, convalescence duration, and treatment costs. Chinese invention patent (application No. 202310284669.7) discloses a sutureless composite-type keratoprosthesis and fabrication method thereof. The invention comprises a biological anterior lamina and a polymeric posterior lamina, wherein sutureless corneal repair is achieved through insertion of fixation wires from the biological anterior lamina into the recipient bed. However, such wire insertion causes secondary trauma, posing risks of stromal scar formation and corneal neovascularization. Moreover, the presence of fixation wires compromises corneal transparency and aesthetics. Currently, none of the existing graft materials can simultaneously replace donor corneas in penetrating keratoplasty and enable sutureless repairing.

### SUMMARY OF THE INVENTION

The purpose of this part is to overview some aspects of embodiments of the present invention and to briefly introduce some preferred embodiments. Simplifications or omissions may be made in this part and in the abstract of description and the title of invention of the present application to avoid blurring the purposes of this part, the abstract of description and the title of invention, and such simplifications or omissions may not be used for limiting the scope of the present invention.

To solve the shortcomings of the prior art, in the first aspect, the present invention provides a decellularized cornea which contains solidified monomer polymer solution; the monomer polymer solution comprises component 1 and component 2, wherein the component 1 is methacryloyl derivatives; the component 2 is selected from one or more of polyethylene glycol diacrylate (PEGDA) or polyethylene glycol methyl ether methacrylate (PEGMA).

In the present invention, under visible light or ultraviolet irradiation, the monomer polymer inside the decellularized cornea undergoes a polymerization reaction and forms a polymeric network in the corneal stroma; the polymeric network has no swelling property or low swelling property, which limits the swelling of the decellularized porcine cornea in a solution.

In the present invention, after the monomer polymer inside the decellularized cornea undergoes a polymerization reaction and forms a polymeric network in the corneal stroma, the light transmittance of the cornea is greater than 70% within a visible spectral region; under normal temperature conditions, after immersing in normal saline for 48 hours, a water absorption rate is 10-35%.

In the present invention, the decellularized cornea is selected from biomaterials suitable for preparation of an artificial cornea, comprising but not limited to corneas, decellularized corneas and transgenic corneas from mammals such as humans, pigs, horses and cows.

A recipient refers to an individual that receives implantation of the decellularized cornea, which may be a vertebrate, preferably a mammal. Examples of mammals include, but are not limited to, humans, non-human primates, mice, rats, dogs, cats, horses, and cows.

In the present invention, the monomer polymer comprises component 1, wherein the component 1 is selected from one or more of methacrylated derivatives; the concentration is 10-50% (w/v).

In a preferred embodiment, methacrylated derivatives include methylpropylated polysaccharides, proteins, or acellular matrices.

In a more preferred embodiment, methacrylated derivatives include gelatin methacryloyl and/or chondroitin sulfate methacrylate.

In the present invention, the component 2 is selected from one or more of polyethylene glycol diacrylate (PEGDA) or polyethylene glycol methyl ether methacrylate (PEGMA); the concentration is 0.001 g/ml-0.5 g/ml.

In a preferred embodiment, the monomer polymer solution is formed by mixing a 10-30% (w/v) component 1 solution with a 0.001 g/ml-0.5 g/ml component 2 solution; preferably, the monomer polymer solution is formed by mixing a 10-30% (w/v) component 1 solution with a 0.05-0.2 g/ml component 2 solution; and a volume ratio of the component 1 solution to the component 2 solution is 10:0-8:2, preferably 9:1-8:2.

In a more preferred embodiment, the monomer polymer solution is formed by mixing a 10-30% (w/v) gelatin methacryloyl and/or chondroitin sulfate methacrylate with a 0.001 g/ml-0.5 g/ml polyethylene glycol diacrylate (PEGDA) or polyethylene glycol methyl ether methacrylate (PEGMA); and a volume ratio of the component 1 solution to the component 2 solution is 10:0-8:2, preferably 9:1-8:2.

In a more preferred embodiment, the monomer polymer solution is formed by mixing a 10-30% (w/v) gelatin methacryloyl with a 0.05 g/ml-0.2 g/ml polyethylene glycol diacrylate (PEGDA); and the volume ratio is 9:1-8:2.

In a more preferred embodiment, the monomer polymer solution is formed by mixing a 20% (w/v) gelatin methacryloyl with a 0.1 g/ml polyethylene glycol diacrylate (PEGDA); and the volume ratio is 9:1-8:2.

Further, the monomer polymer solution comprises a photoinitiator or a temperature initiator.

In a preferred embodiment, the photoinitiator is a blue light or ultraviolet light initiator, and comprises lithium phenyl (2,4,6-trimethylbenzoyl) phosphinate (LAP); and the temperature initiator comprises azodiisobutyronitrile (AIBN).

In a preferred embodiment, the concentration of the photoinitiator is 0.1%-0.5%; and the concentration of the temperature initiator is 0.1-0.2 mol/L.

In a preferred embodiment, the condition to maintain the solidified monomer polymer solution is 4-25° C.; preferably 4-20° C.

In a preferred embodiment, the condition for liquefaction is surface temperature surpassing 25° C., preferably 35-38° C.

110 In a preferred embodiment, the wavelength range of the visible light is 300-800 nm, the energy of the light ranges from 100-500 mW/cm², and the irradiation time ranges from 30 seconds to 2 minutes; more preferably, the wavelength range is 365-405 nm, and the irradiation time is 30-60 seconds.

In the second aspect, the present invention provides a method for preparing a decellularized cornea, comprising:
immersing the decellularized cornea in a monomer polymer solution, taking out the decellularized cornea, then activating a polymerization reaction of the monomer polymer in the decellularized cornea.

In the present invention, the monomer polymer solution comprises methacrylated derivatives with a concentration of 10-50%

In a preferred embodiment, methacrylated derivatives include methacrylated polysaccharides, proteins, or acellular matrices.

In a more preferred embodiment, methacrylated derivatives includes gelatin methacryloyl and/or chondroitin sulfate methacrylate.

In a preferred embodiment, the monomer polymer solution further comprises one or more of polyethylene glycol diacrylate (PEGDA) or polyethylene glycol methyl ether methacrylate (PEGMA) with a concentration of 0.001 g/ml-1 g/ml.

In a preferred embodiment, the monomer polymer solution is formed by mixing a 10-30% (w/v) component 1 solution with a 0.001 g/ml-0.5 g/ml component 2 solution; preferably, the monomer polymer solution is formed by mixing a 10-30% (w/v) component 1 solution with a 0.05-0.2 g/ml component 2 solution; and a volume ratio of the component 1 solution to the component 2 solution is 10:0-8:2, preferably 9:1-8:2.

In a more preferred embodiment, the monomer polymer solution comprises component 1: a 10-30% (w/v) gelatin methacryloyl and/or chondroitin sulfate methacrylate; component 2: a 0.001 g/ml-0.5 g/ml polyethylene glycol diacrylate (PEGDA) and/or polyethylene glycol methyl ether methacrylate (PEGMA); and a volume ratio of the component 1 solution to the component 2 solution is 10:0-8:2, preferably 9:1-8:2.

In a more preferred embodiment, the monomer polymer solution is formed by mixing a 10-30% (w/v) gelatin methacryloyl with a 0.05 g/ml-0.2 g/ml polyethylene glycol diacrylate (PEGDA); and the volume ratio is 9:1-8:2.

In a more preferred embodiment, the monomer polymer solution is formed by mixing a 20% (w/v) gelatin methacryloyl with a 0.1 g/ml polyethylene glycol diacrylate (PEGDA); and the volume ratio is 9:1-8:2.

In the present invention, the monomer polymer solution may further includes anti-inflammatory drugs, antibiotics, and/or substances for improving biocompatibility, such as voriconazole, chondroitin sulfate methacrylate, or methacrylated derivatives.

Further, the monomer polymer solution comprises a photoinitiator or a temperature initiator.

In a preferred embodiment, the photoinitiator is a blue light or ultraviolet light initiator, and comprises lithium phenyl (2,4,6-trimethylbenzoyl) phosphinate (LAP); and the temperature initiator comprises azodiisobutyronitrile (AIBN).

In a preferred embodiment, the concentration of the photoinitiator is 0.1%-0.5%; and the concentration of the temperature initiator is 0.1-0.2 mol/L. When light activation is used, the wavelength is 300 nm-600 nm, and the activation time is 30 seconds to 2 minutes; and when the temperature activation is used, temperature is above 60° C. and the activation time is 2 hours to 12 hours.

In a preferred embodiment, a preparation method of the monomer polymer solution is:
a) preparing a 10-30% (w/v) methacrylated derivatives solution by using 0.1-0.5% LAP solution, to obtain the component 1 solution;
b) preparing a PEGDA solution of 0.001 g/ml-0.5 g/ml to obtain the component 2 solution;
c) mixing the component 1 solution with the component 2 solution in a volume ratio of 10:0-8:2.

In the present invention, in an immersion process, the immersing or soaking process can be accelerated by ultrasonic, vacuuming or stirring at the same time to facilitate the penetration of monomer polymer into the stroma of the decellularized cornea.

In a preferred embodiment, the operation mode of the immersion is to immerse the decellularized cornea in a monomer polymer solution of at least 10 times the volume and oscillating by a shaker for 18-48 hours.

Further, the method disclosed by the present invention further comprises a cooling step, including immersing the decellularized cornea into a monomer polymer solution, taking out the decellularized cornea and then cooling to solidify the monomeric polymer in the decellularized cornea.

In a preferred embodiment, the cooling method comprises placing the decellularized cornea in an environment of 4° C. for 15-30 minutes, and then storing the decellularized cornea in an environment of 4-25° C.; preferably storing the decellularized cornea in an environment of 4-20° C.

In the above preparation method provided by the present invention, preferably all operations are conducted in a sterile ultra-clean environment.

Further, the method of the present invention further comprises a post-treatment step of solidification, comprising removing the monomer polymer solution on the surface of the decellularized cornea; conventional operations can be used, such as rinsing, wiping, and the like. Taking the decellularized cornea as an example, the removal method may be using tweezers to clamp the decellularized cornea, wiping against the surface of the dust-free absorbent paper, and removing excessive monomer polymer solution; and further comprising sterilizing the product, cutting the product into different sizes of implants, and the like.

In the present disclosure, the decellularized cornea includes, but is not limited to, biological tissues, acellular scaffold materials, transgenic organs, transgenic tissues, cell products and derivatives thereof, and artificial biomimetic materials.

Further, the method in the present invention also comprises a pre-treatment step of treating the decellularized cornea, comprising femtosecond cutting to obtain a corneal stroma lamella with a thickness of 100-400 μm and a diameter of 9 mm; epithelial and endothelial curettage, decellularization treatment and virus inactivation to obtain the decellularized cornea comprising a Bowman's layer and a corneal stroma layer.

In the third aspect, the present invention provides a decellularized cornea obtained by the above preparation method.

In the present invention, a solidified monomer polymer solution is contained in and/or attached to the surface of the decellularized cornea, when it is applied to the recipient cornea, the monomer polymer solution liquefies, and partially seeps out of the decellularized cornea and then immersing between the recipient cornea and the decellularized cornea, and in the recipient cornea; under the irradiation of visible light or ultraviolet rays, the monomer polymer undergoes a polymerization reaction, and an adhesion layer is formed between the decellularized cornea and the recipient cornea.

In the present invention, a recipient refers to an individual that receives an implanted decellularized cornea, and may be a vertebrate, preferably a mammal. And the mammal may be human, non-human primates, mice, rats, dogs, cats, horses, or cows, but are not limited to these examples.

In the present invention, the adhesion layer is composed of an aggregation network formed by methacrylated derivatives (methacrylated gelatin, chondroitin sulfate methacrylate, etc.) between the recipient cornea and the stroma network of the decellularized cornea.

In a preferred embodiment, the condition for holding the solidified monomer polymer solution is 4-25° C.

In a preferred embodiment, the liquefaction condition is that the surface temperature is greater than or equal to 25° C., preferably 35-38° C.

In a preferred embodiment, the wavelength range of the visible light is 300-800 nm, the energy of the light ranges from 100-500 mW/cm², and the irradiation time ranges from 30 seconds to 2 minutes; more preferably, the wavelength range is 365-405 nm, and the irradiation time is 30-60 seconds.

In the fourth aspect, the present invention provides an method for preparing the decellularized cornea and/or the decellularized cornea disclosed above, and the applications in preparation of a corneal donor, a corneal graft, a tissue substitute material, a tissue sealant, a medical dressing, or a drug carrier.

### Advantageous Effects

The present invention utilizes the unique thermoresponsive property of methacrylated gelatin (GelMA), which solidifies at 4-20° C. and rapidly liquefies upon warming to 35-38° C. This enables the GelMA pre-incorporated into the biomaterial matrix network to liquefy and exude rapidly when applied to the body surface, followed by photoactivated polymerization to form an adhesive network for graft fixation. By blending GelMA with other polymerizable monomers at a specific ratio, the resulting composite retains the temperature-sensitive phase transition behavior while significantly improving the resistance to degradation, adhesive strength, and swelling properties of the bonding layer. The low-swelling decellularized corneal graft prepared by this invention exhibits thermosensitive and photo-polymerizable characteristics, along with abundant material availability and sutureless surgical procedures for rapid trauma repair. Notably, the decellularized cornea provided herein can directly contact aqueous humor without risk of hydration-induced swelling, making it suitable for replacing corneal stroma and applicable to penetrating keratoplasty.

### BRIEF DESCRIPTION OF THE DRAWINGS

FIG. 1 shows physical states of monomer polymer solutions with different proportions.

FIG. 2 shows viscosity of monomer polymer solutions with different proportions.

FIG. 3 shows cell compatibility after polymerization of monomer polymers with different proportions.

FIG. 4 shows monomer polymers immersed in decellularized porcine corneal stroma.

FIG. 5 shows the temperature-dependent rheological properties of a low-swelling (composite) decellularized porcine cornea and a conventional decellularized porcine cornea

FIG. 6 shows the rheological properties of a low-swelling (composite) decellularized porcine cornea and a conventional decellularized porcine cornea before and after 405 nm light activation

FIG. 7 shows the tensile strength comparison between a low-swelling (composite) decellularized porcine cornea and a conventional decellularized porcine cornea

FIG. 8 shows changes of corneal transparency before and after swelling.

FIG. 9 shows the water absorption rate of cornea before and after swelling.

FIG. 10 shows the transmittance of cornea before and after swelling.

FIG. 11 shows the Young's modulus of cornea before and after low swelling treatment.

FIG. 12 shows the mechanical performance of cornea before and after low swelling treatment.

FIG. 13 shows the schematic diagram of interface strength testing for low-swelling (composite) decellularized porcine cornea.

FIG. 14 shows the interface strength comparison between low-swelling (composite) decellularized porcine cornea and medical-grade bioadhesives.

FIG. 15 shows sutureless repair of corneal stroma defect using low-swelling (composite) decellularized porcine cornea.

FIG. 16 shows the gross photographs, AS-OCT images and thickness scanning results before and after corneal stromal repairing using low-swelling (composite) decellularized porcine cornea.

FIG. 17 shows the steady adhesion formed between a low-swelling (composite) decellularized porcine cornea and a natural cornea.

FIG. 18 shows the histological staining micrograph of the adhesive layer formation of a low-swelling (composite) decellularized porcine cornea.

FIG. 19 shows the results of slit lamp, fluorescein staining, AS-OCT and thickness scanning in an in vivo animal experiment.

FIG. 20 shows comparison effects between a low-swelling (composite) decellularized porcine cornea and a donor cornea after transplantation.

FIG. 21 shows comparison effects between a low-swelling (composite) decellularized porcine cornea and a conventional decellularized porcine cornea after transplantation.

### DETAILED DESCRIPTION

The technical solution in the embodiments of the present invention will be clearly and fully described below in combination with the drawings in the embodiments of the present invention. Apparently, the described embodiments are part of the embodiments of the present invention, not all of the embodiments. Based on the embodiments in the present invention, all other embodiments obtained by those skilled in the art without contributing creative labor will belong to the protection scope of the present invention.

Although steps, substances or materials, and reaction conditions similar to or equivalent to those disclosed herein may be used in the implementation process of the present invention, preferred steps, substances or materials, and reaction conditions are described herein.

When a valve range is described herein, the range is intended to include the end values and all integers and fractions within the range unless otherwise specified, and all values within the range can realize the effect of the present invention.

Unless otherwise specified, all technical and scientific terms and abbreviations used herein have the meanings generally understood by those ordinary skilled in the field of the present invention or in the application field of the terms.

The singular forms of words used herein include the plural, and vice versa. Thus, “a”, “an” and “the” generally comprises the plurals of the corresponding terms. “An embodiment” or “embodiment” used herein means a specific feature, structure or characteristic that may be included in at least one implementation of the present invention. “In an embodiment” appearing in different places in the description does not mean the same embodiment, nor a separate or selective embodiment that is mutually exclusive with other embodiments.

“Comprise”, “have”, “include” or “contain” used herein means inclusive or open, and does not exclude additional, unquoted materials or method steps.

“None”, “low” or “reduce” used herein means a state in which reduction or disappearance is produced, or reduction in a detectable or observed amount. In some implementation solutions, this decrease or reduction or disappearance is measured by using one of assessment tools described herein. In some implementation solutions, none or decrease or reduction indicates a difference.

Swelling refers to a phenomenon that the volume of a polymer expands and becomes larger in a solvent.

Swelling property refers to the imbibition performance of a substance in a solvent, generally expressed by a weight increase percent or imbibition rate and water absorption rate. “Low swelling property” or “reducing swelling property” used herein means a phenomenon that the volume of a substance (cornea) is increased when contacting or immersing in a solvent (aqueous humor) or a state in which a tendency becomes decreased or reduced, or the amount of observable or measurable swelling is decreased or reduced, or a difference from decrease or reduction.

Decellularized cornea: a cornea from a mammal such as a human, a pig, a horse or a cow is treated with a conventional decellularization method (including repeated freezing and thawing, high and low osmotic pressure treatment, surfactant treatment, hyperstatic pressure treatment, nuclease treatment and phospholipase treatment) to remove stromal cells and endothelial cells, eventually obtaining a decellularized cornea.

Decellularized porcine cornea is prepared by virus inactivation and decellularization technology, and comprises a Bowman's layer and a corneal stroma layer.

Low-swelling decellularized cornea and the composite decellularized cornea have the same meaning and all refer to the decellularized cornea disclosed in the present invention.

Experimental methods in which specific conditions are not specified in the embodiment are carried out under conventional conditions or as recommended by the manufacturer. The reagents or instruments used of which the manufacturers are not specified are all conventional products that can be purchased commercially.

Sources of part of materials in embodiments of the present invention:

Fresh porcine cornea: non-denatured fresh porcine cornea purchased from the market.

SDS solution: sodium dodecyl sulfate solution, purchased from Beijing Solarbio Science & Technology Co., Ltd., with an item number of S8010.

Nuclease: deoxyribonuclease, purchased from Sino Biological Inc., with an item number of SSNP01.

330 Photoinitiator (LAP) lithium phenyl (2,4,6-trimethylbenzoyl) phosphinate, purchased from Shanghai Yuanye Bio-Technology Co., Ltd., with an item number of Y43995.

Gelatin methacryloyl (GelMA): a gelatin derivative obtained by the reaction of gelatin and methacrylic anhydride, purchased from Suzhou Yongqinquan Intelligent Equipment Co., Ltd., with an item number of EFL-GM-90.

PEGDA solution: polyethylene glycol diacrylate solution, purchased from Sigma, with an item number of 455008.

PBS: phosphate buffer solution, purchased from Beijing Solarbio Science & Technology Co., Ltd., with an item number of P1020.

Rabbit: purchased from Jinan Xilingjiao Breeding Center.

### Embodiment 1 Physical Properties of Gelatin Methacryloyl and a Mixture Thereof

PEGDA and PEGMA polymers have low swelling properties and can limit the swelling of DPC in an aqueous solution after forming a polymerization network in a decellularized porcine cornea (DPC). However, it is difficult for cells to adhere to the surfaces of PEGDA and PEGMA polymers. Therefore, the mixing of methacryloyl derivatives such as gelatin methacryloyl/methylpropenylated chondroitin sulfate with good biocompatibility with PEGDA and PEGMA in specific proportions can improve the cell adhesion of PEGDA and PEGMA.

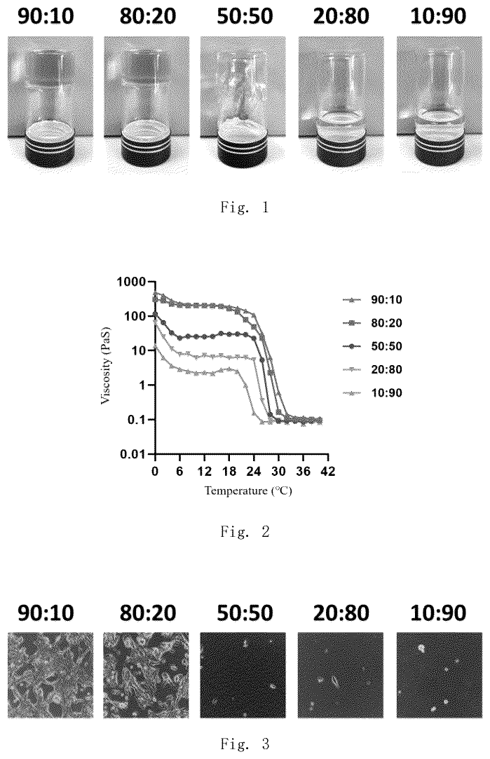

After 20% GelMA (gelatin methacryloyl) is mixed with 0.1 g/ml PEGMA solution with an average mass fraction of 700 in different proportions, physical states, viscosity, transmittance, a water absorption rate and cell adhesion are analyzed. The results are shown in Table 1, FIG. 1 and FIG. 2. When the proportion of 20% GelMA is greater than 20%, the state is a solid state at 4-20° C. and a liquid state at 37° C. As the proportion of GelMA is increased, the viscosity is increased, the transmittance is decreased, and the water absorption rate is increased. When the proportion of 20% GelMA is greater than 80%, good cell adhesion is presented after gelation.

TABLE 1

| | Physical | Physical | | | 24 h Water | |
|---|---|---|---|---|---|---|
| | State | State | Viscosity | Transmittance | Absorption | Cell |
| PEGMA:GelMA | 4°-20° | 37° | (mPa · s) | (%) | Rate (%) | Adhesion |
| PEGDA | Liquid | Liquid | 6.8 ± 0.1 | 61-75% | 107.7 ± 1.3 | Inadhesion |
| 90:10 | Liquid | Liquid | 9.6 ± 0.2 | 62-74% | 108.6 ± 1.1 | Inadhesion |
| 80:20 | Solid | Liquid | 7.3 ± 0.1 | 63-76% | 110.8 ± 1.5 | Inadhesion |
| 50:50 | Solid | Liquid | 5.4 ± 0.8 | 60-75% | 113.3 ± 3.1 | Inadhesion |
| 20:80 | Solid | Liquid | 8.2 ± 0.1 | 51-73% | 120.3 ± 2.9 | Adhesion |
| 10:90 | Solid | Liquid | 26.9 ± 1.6 | 43-68% | 123.2 ± 0.6 | Adhesion |
| GelMA | Solid | Liquid | 31.3 ± 1.2 | 41-63% | 127.3 ± 0.5 | Adhesion |

### Embodiment 2 Cytocompatibility of a Mixture of PEGDA and Gelatin Methacryloyl in Different Proportions

0.1 g/ml PEGDA solution and 20% gelatin methacryloyl solution are fully mixed at the ratios of 9:1, 8:2, 5:5, 2:8 and 1:9 respectively, and 1 ml of mixture is added to each well of a 6-well cell culture plate at 37° C. to cure the liquid by photocuring. Corneal epithelial cells are inoculated in the 6-well plate and cultured in cell culture fluid in a carbon dioxide incubator. After 72 hours, the adherence and the growth state of the cells are observed under a cell microscope. Results are shown in FIG. 3. When PEGDA is mixed with gelatin methacryloyl at the ratios of 2:8 and 1:9, the corneal epithelial cells can adhere to the gel surface. The gelatin prepared by the mixed solution in other proportions does not make the corneal epithelial cells adhere.

### Embodiment 3 Preparation of Low-Swelling (Composite) Decellularized Porcine Cornea

(1) Preparation of decellularized porcine cornea: the epithelium and the endothelium are scraped from a fresh porcine cornea, immersed in 0.5% SDS solution, treated with 500 U/ml deoxyribonuclease for 2 hours, and then washed with sufficient PBS for 6-8 times.

(2) Preparation of methacrylate compound solution: 20% gelatin methacryloyl solution and 0.1 g/ml PEGDA (PEGDA with an average relative molecular weight of 700) solution are prepared by 0.3% LAP solution. The gelatin methacryloyl solution is fully mixed with the PEGDA solution in a volume ratio of 8:2-10:0.

(3) Composite gelatin methacryloyl: the decellularized porcine cornea is immersed in the gelatin methacryloyl solution, then put into a constant temperature shaker at 37° C., and oscillated for 24 hours at 120 RPM. After immunofluorescence assay for 24 hours, the monomer polymer is fully immersed in the decellularized porcine corneal stroma (FIG. 4).

(4) Acquirement of a low-swelling decellularized porcine cornea: the decellularized porcine cornea is taken out, the surface solution is removed and then the decellularized porcine cornea is put under 4-25° C. (for example, 4° C., 10° C., 15° C., 20° C. and 25° C.) for cooling down and solidifying the gelatin methacryloyl.

### Embodiment 4 Preparation of Decellularized Porcine Cornea

(1) Preparation of decellularized porcine cornea: Fresh porcine corneas are stripped of the epithelium and endothelium, then immersed in 0.5% SDS solution. After treatment with 500 U/ml nucleases for 2 hours, the corneas are thoroughly rinsed with balanced salt solution (BSS) 6-8 times.

(2) Preparation of voriconazole-methacrylated gelatin (GelMA) mixture: A 30% GelMA solution is prepared using 0.5% LAP. Voriconazole powder (5 mg) is first weighed and then dissolved in 10 ml of dimethyl sulfoxide (DMSO) to prepare a 500 μg/ml stock solution. The GelMA solution is then mixed with the voriconazole solution at a 9:1 volumetric ratio (v/v).

(3) Fabrication of voriconazole-loaded decellularized porcine cornea: The decellularized corneas are immersed in the voriconazole-GelMA mixture and incubated in a 37° C. constant-temperature shaker at 120 rpm for 24 hours. Subsequently, the corneas are taken out, cleaned off solution on the surface, and cooled at 4° C.

### Test Example 1 Rheological Properties, Storage Modulus and Loss Modulus

The rheological properties of the low-swelling decellularized porcine cornea (Embodiment 3) and conventional decellularized porcine cornea are evaluated under varying temperatures using a rheometer. As shown in FIG. 5, the low-swelling decellularized porcine cornea maintain stable rheological behavior below 20° C. When above 20° C., its storage modulus (G′) begins to decline, reaching a minimum at about 27° C. before rebounding. This phenomenon correlates with the temperature-dependent phase transition of methacrylated gelatin (GelMA) within the low-swelling decellularized porcine cornea, where GelMA transitions from a gel to a liquid phase with exudation above 20° C. In contrast, the conventional decellularized cornea exhibits no significant rheological changes across the tested range (0-40° C.), confirming the thermo-responsiveness of the low-swelling decellularized porcine cornea.

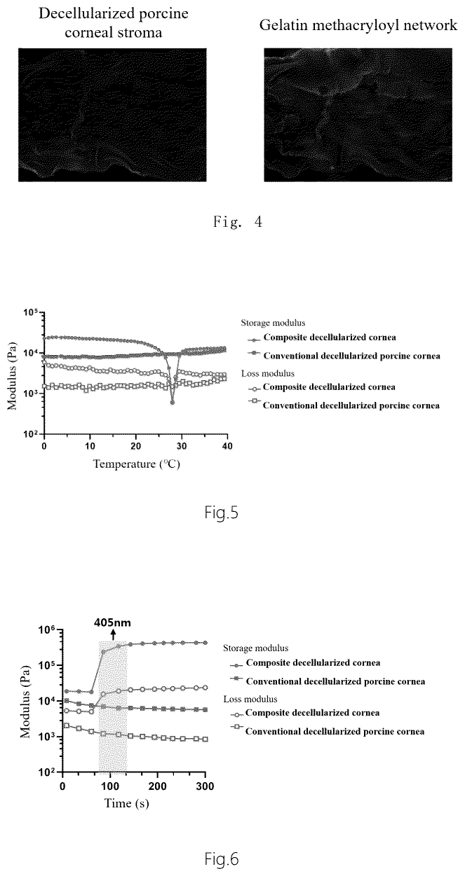

Subsequently, both low-swelling decellularized porcine cornea and conventional decellularized porcine cornea are irradiated with a 405 nm light source. The low-swelling decellularized porcine cornea is demonstrated a marked alterations in rheological behavior: its storage modulus (G′) increases significantly, while the loss modulus (G″) decreases, resulting in a widened gap between G′ and G″ (FIG. 6). No light-induced changes are observed in the conventional graft, verifying the photosensitivity of the low-swelling variant.

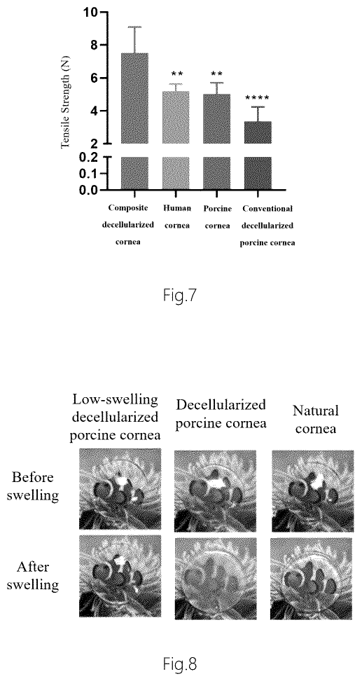

Finally, tensile testing reveals mechanical performance differences of low-swelling decellularized porcine cornea and conventional decellularized porcine cornea (FIG. 7). The low-swelling graft exhibits a mean tensile strength of 7.52 N, surpassing both native human corneas (5.20 N) and porcine corneas (5.03 N). This enhancement stems from the polymer network crosslinking within the stromal matrix, which reinforces structural integrity. Conversely, the mean tensile strength of the conventional decellularized cornea is 3.36 N, lower than low-swelling decellularized porcine cornea and native corneas, due to collagen fiber damage during decellularization.

### Test Example 2 Mechanical Performance, Swelling Resistance and Light Transmittance

Prepare a decellularized porcine cornea according to the method described in Embodiment 3, wherein the gelatin methacryloyl solution is mixed with the PEGDA solution in a volume ratio of 9:1, and the obtained decellularized porcine cornea is irradiated with 365 nm ultraviolet light for 2 minutes for photocuring. Then the decellularized porcine cornea, an untreated normal decellularized porcine cornea and a natural cornea (fresh porcine cornea) are simultaneously immersed in a balanced salt solution (normal saline), placed at room temperature for 48 hours, and then photographed to observe and contrast the changes in the water absorption rate and transparency of the corneas before and after immersion.

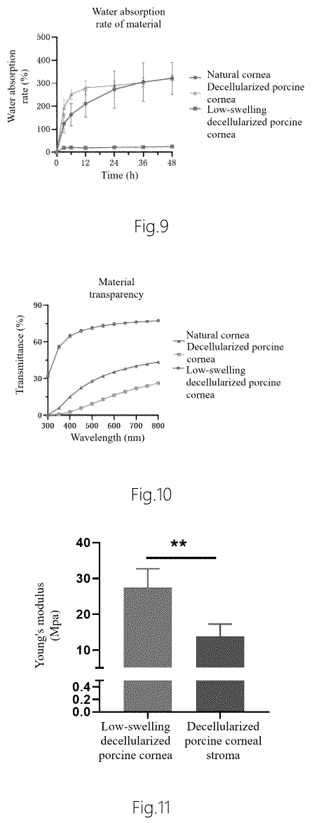

Results shows that the low-swelling decellularized porcine cornea has a low swelling property, after immersing in normal saline for 48 hours, the cornea remains transparent without edema, while the untreated decellularized porcine cornea has obvious edema and the transparency is decreased (FIG. 8). The water absorption rate of the low-swelling decellularized porcine cornea is 25±4%, the water absorption rate of the normal decellularized porcine cornea is 324±12%, and the water absorption rate of the natural cornea is 322±70% (FIG. 9). Through spectrophotometer analysis, the transmittance of the low-swelling decellularized porcine cornea is 77±1.0%, the transmittance of the normal decellularized porcine cornea is 26±1.3%, and the transmittance of the natural cornea is 43±0.2% (FIG. 10).

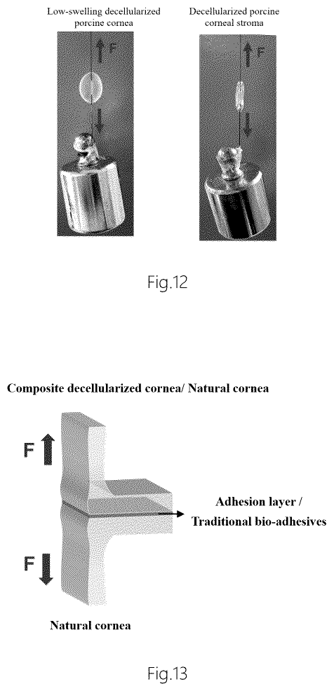

The low-swelling decellularized porcine cornea has a higher Young's modulus than the normal decellularized corneal stroma, and shows better mechanical performance (FIGS. 11 and FIG. 12). The increase of the mechanical performance and the swelling resistance of the cornea proves that the monomer polymer is infiltrated into the corneal stroma fibers and then polymerized through photo stimulation to form a new polymerization network in the corneal stroma.

### Test Example 3 Adhesion and Interface Strength

The commonly used biological adhesives in clinical practice currently include fibrin glue, alpha cyanoacrylate glue, and PEG based bio-adhesives. The clinical application of PEG based bio-adhesives are greatly limited. Although fibrin adhesives are natural biomaterials and possess good biocompatibility, their bonding strengths are weak. Meanwhile, their raw materials origin from allogeneic tissues, which poses a risk of virus transmission. Alpha cyanoacrylate adhesives have high adhesive strengths, but their disadvantages are also very obvious. Alpha cyanoacrylate adhesives generate polymerization heat during use, causing thermal damage to the adhesive tissue. In addition, the material formed after solidification is relatively brittle and has poor mechanical compatibility with soft tissues, and produces foreign body reactions that hinder wound healing. PEG bio-adhesives easily swell and cause wound dehiscence after use. In addition, PEG gelation is not conducive to cell adhesion and hinders wound repair. Compared with representative bio-adhesives used in traditional applications, low swelling decellularized porcine corneas can achieve biological adhesion with recipient tissues without the need for bio-adhesives, with higher operability, biocompatibility, and advantages in promoting wound healing.

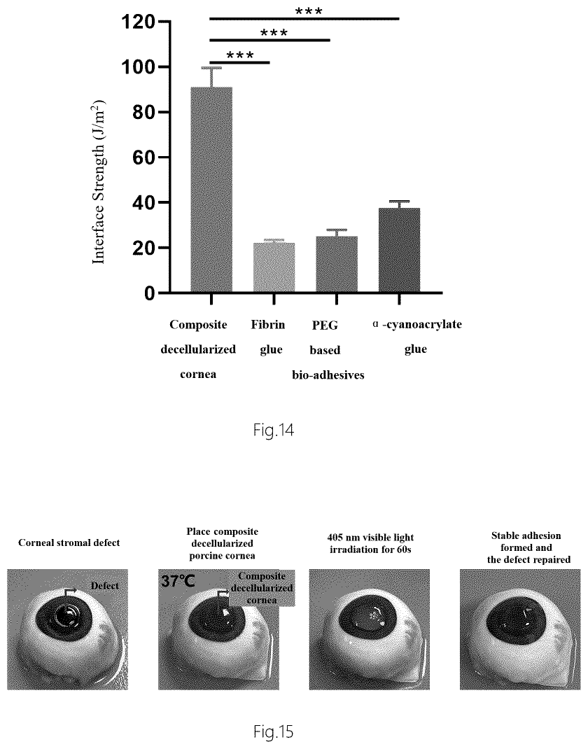

By testing the adhesive strength of the low swelling decellularized porcine cornea prepared in Embodiment 3 and the traditional bio-adhesives to the surface of natural cornea (FIG. 13), the results show that the low swelling decellularized porcine cornea could form an interface strength of 91.1 J/m² with the natural cornea. The interface strength of fibrin glue (Guangzhou Beixiu Biotechnology Co., Ltd.) is 22.3 J/m², the interface strength of PEG glue (absorbable dura mater sealing medical glue Success Biotechnology Co., Ltd.) is 25.1 J/m², and the interface strength of α-cyanoacrylate glue (Guangzhou Baiyun Medical Adhesive Co., Ltd.) is 38.6 J/m². The interfacial strength of low swelling decellularized porcine cornea on natural corneal surface is significantly higher than that of traditional biological adhesives (FIG. 14).

### Application Example 1 In Vitro Experiment-In Situ Solidification

(1) A circular corneal stromal defect with a diameter of 6 mm and a depth of 500 μm is created on the porcine corneal surface using a trephine and lamellar knife.

(2) A low-swelling decellularized porcine corneal graft prepared according to the method of Embodiment 3 (wherein methacrylated gelatin solution and PEGDA solution mixed at 8:2 v/v ratio) is placed onto the defect. Then raise ambient temperature to 37° C., followed by 405 nm visible light irradiation for 60 seconds (FIG. 15).

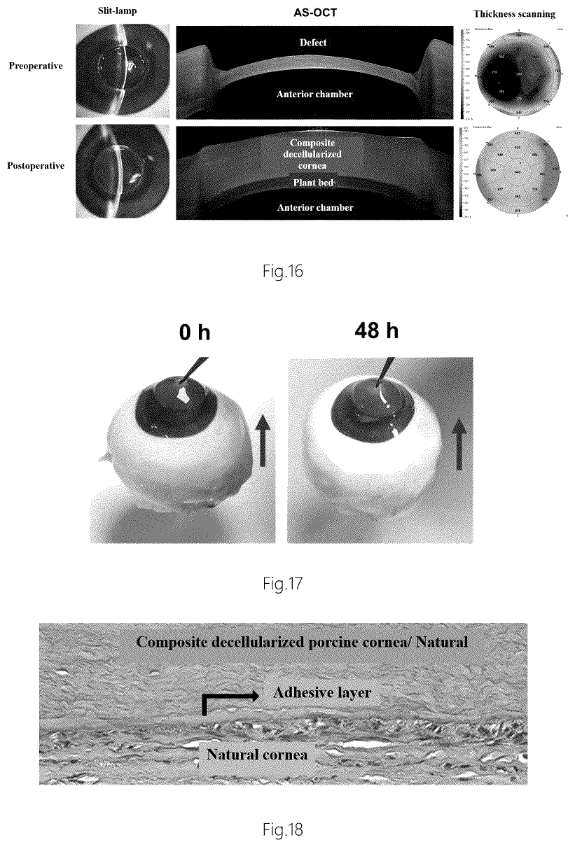

(3) Post-repair slit-lamp examination confirms successful defect closure. Anterior segment optical coherence tomography (AS-OCT) and pachymetry demonstrate complete stromal defect filling and restored corneal curvature (FIG. 16).

(4) Forceps gripping tests reveal robust adhesion: the composite graft adheres firmly to the native cornea, enabling whole-eye lifting. Stable adhesion persists for 48 hours post-implantation (FIG. 17).

(5) Histological section staining was performed on the adhered corneas. The H&E staining results revealed a polymerization reaction occurring between the composite decellularized porcine cornea and the recipient cornea, leading to the formation of an adhesive layer (FIG. 18).

### Application Example 2 In Vivo Experiment-In Situ Solidification

(1) Create a circular corneal stromal defect with a diameter of 6 mm and a depth of 200 μm on the surface of the rabbit cornea using a trephine and a lamellar knife.

(2) In situ solidification: place low swelling decellularized porcine corneas prepared according to the method of Embodiment 3 (wherein methacrylated gelatin solution and PEGDA solution are in a volume ratio of 8:2) of equal size and thickness onto the corneal defect site, and then irradiate with 405 nm visible light for 60 seconds.

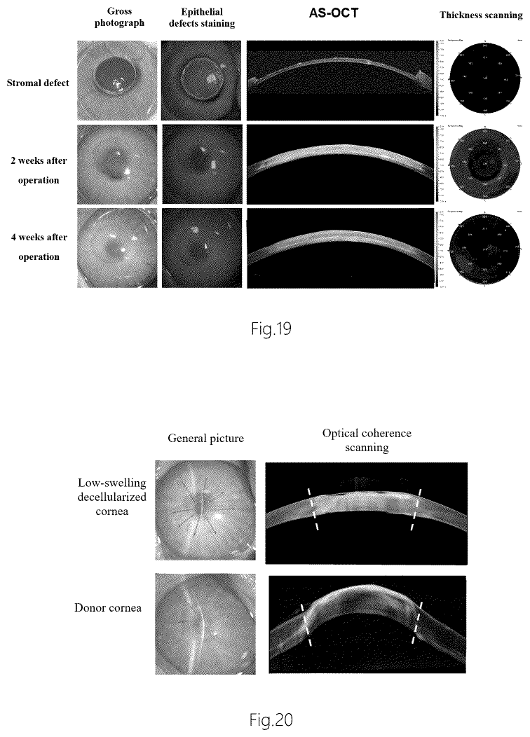

(3) Perform a check-up every two weeks after surgery. Two weeks after surgery, the graft remains stable without displacement or detachment; staining of epithelial defects proves that the corneal epithelium is fully repaired within 2 weeks; the OCT and thickness scanning results showed that the corneal defect is successfully repaired and with stroma regeneration (FIG. 19).

### Application Example 3 In Vivo Experiment-Suture

A penetrating defect with a diameter of 3 mm is made in the center of the rabbit cornea. Penetrating keratoplasty is performed subsequently. The low-swelling decellularized porcine cornea prepared according to the method described in Embodiment 3 (wherein the methacrylated gelatin solution is mixed with the PEGDA solution in a volume ratio of 9:1), and a donor cornea with a healthy endothelium (a cornea allograft taken from the eyeball of another rabbit) is implanted respectively, and then the corneal perforation is repaired by breakpoint suture (FIG. 20). After implant the low-swelling decellularized porcine cornea as a graft, on the 5th day after the penetrating keratoplasty, the graft is stable and has no edema and the transparency of the cornea is good. On the 14th day after the penetrating keratoplasty, the corneal epithelium is repaired, the surgical irritant inflammation is reduced, and the graft has no edema and no immunological rejection. On the 18th day after the penetrating keratoplasty, the cornea is stable, the graft is fused with an implant bed, the anterior chamber is stable, and aqueous humor does not leak. On the 28th day after the penetrating keratoplasty, the repair effect of the low-swelling decellularized porcine cornea is comparable to that of the donor cornea, the cornea has no edema and good transparency, the anterior chamber is stable and the graft is biologically fused with the implant bed.

### Application Example 4 In Vivo Experiment-Suture

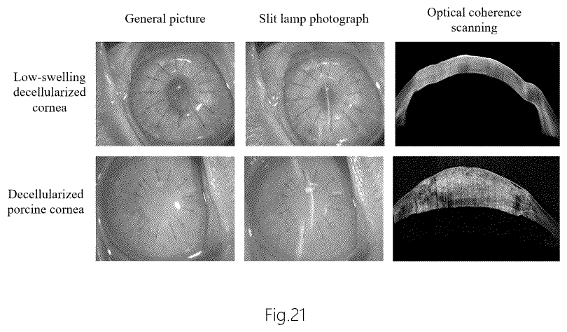

A penetrating defect with a diameter of 6 mm is made in the center of the rabbit cornea. Penetrating keratoplasty is performed subsequently. The low-swelling decellularized porcine cornea (a graft with a diameter of 6 mm is cut in advance by a corneal trephine) prepared according to the method described in Embodiment 3 (wherein the gelatin methacryloyl solution is fully mixed with the PEGDA solution in a volume ratio of 9:1), and an ordinary decellularized porcine cornea is implanted respectively by breakpoint suture (FIG. 21). On the 28th day after the penetrating keratoplasty, the low-swelling decellularized porcine cornea which implanted as a graft, remains transparent, the anterior chamber is stable, and there is no inflammatory irritation. The ordinary decellularized porcine cornea completely loses transparency, turns white and completely covers the pupil area, and the eye loses the visual function. Optical coherence scanning shows that the low-swelling decellularized porcine cornea does not swell, has a thickness equivalent to that of a recipient cornea, and is biologically fused with the recipient cornea. The normal decellularized porcine cornea has severe edema, and has a thickness significantly higher than that of the recipient cornea.

The embodiments of the present invention are introduced above in detail. Specific individual cases are applied herein for elaborating the principle and embodiments of the present invention. The illustration of the above embodiments is merely used for helping to understand the method of the present invention and the core thought thereof. At the same time, changes or variations made by those skilled in the art according to the idea of the present invention and based on the specific embodiments and the application scope of the present invention belong to the protection scope of the present invention. In conclusion, the contents of the description shall not be interpreted as a limitation to the present invention.

1. A decellularized cornea, characterized in that: the interior of the decellularized cornea contains a solidified monomer polymer solution, which comprises component 1 and component 2, wherein component 1 is a methacrylated derivative; component 2 is selected from one or more of polyethylene glycol diacrylate (PEGDA) or polyethylene glycol methyl ether methacrylate (PEGMA).

2. The decellularized cornea according to claim 1, wherein: upon irradiation with visible light or ultraviolet light, the monomer polymer inside the decellularized cornea undergoes a polymerization reaction to form a polymer network in the corneal stroma; the polymer network has non-swelling or low-swelling properties, thereby limiting the swelling of the decellularized cornea in solution.

3. The decellularized cornea according to claim 2, wherein: in the visible light region, the light transmittance is greater than 70%; after soaking in physiological saline for 48 hours at room temperature, the water absorption rate is 10-35%.

4. The decellularized cornea according to claim 1, wherein: component 1 is a 10-50% (w/v) methacrylated derivative; component 2 is a 0.001 g/ml-1 g/ml polyethylene glycol diacrylate or polyethylene glycol methyl ether methacrylate or a mixture thereof; the monomer polymer solution is formed by mixing component 1 and component 2, and the volume ratio of the component 1 solution to the component 2 solution is 10:0-8:2; wherein the methacrylated derivative comprises gelatin methacryloyl or chondroitin sulfate methacrylate or a mixture thereof.

5. The decellularized cornea according to claim 1, wherein: the monomer polymer solution further comprises a photoinitiator or a temperature initiator; the concentration of the photoinitiator is 0.1%-0.5%, and the concentration of the temperature initiator is 0.1-0.2 mol/L;
the photoinitiator is a blue light or ultraviolet light initiator, including lithium 2,4,6-trimethylbenzoylphosphinate; the temperature initiator includes azodiisobutyronitrile.

6. (canceled)

7. A method for preparing a decellularized cornea, comprising: immersing a decellularized cornea in a monomer polymer solution, taking out the decellularized cornea, and then activating the polymerization reaction of the monomer polymer inside the decellularized cornea wherein immersing the decellularized cornea in the monomer polymer solution, occurs by taking out the decellularized cornea and cooling it to solidify the monomer polymer in the decellularized cornea, and then activating the polymerization reaction of the monomer polymer inside the decellularized cornea; the monomer polymer solution comprises component 1: a methacrylated derivative with a concentration of 10-50% (w/v).

8. The preparation method according to claim 7, wherein: the monomer polymer solution further comprises component 2: a polyethylene glycol diacrylate (PEGDA) or polyethylene glycol methyl ether methacrylate (PEGMA) or a mixture thereof with a concentration of 0.001 g/ml-1 g/ml; the volume ratio of component 1 to component 2 is 10:0-8:2.

9. The preparation method according to claim 7, wherein: the monomer polymer solution comprises component 1:10-30% (w/v) gelatin methacryloyl and/or chondroitin sulfate methacrylate; component 2:0.001 g/ml-0.5 g/ml polyethylene glycol diacrylate (PEGDA) and/or polyethylene glycol methyl ether methacrylate (PEGMA); the volume ratio of component 1 to component 2 is 10:0-8:2.

10-12. (canceled)

13. A method for preparing a decellularized cornea, comprising: immersing a decellularized cornea in a monomer polymer solution, taking out the decellularized cornea, and then activating the polymerization reaction of the monomer polymer inside the decellularized cornea wherein the preparation process of the monomer polymer solution is:
a) preparing a 10-50% (w/v) methacrylated derivative solution with a 0.1-0.5% LAP solution to obtain a component 1 solution;
b) preparing a 0.001 g/ml-1 g/ml polyethylene glycol diacrylate or polyethylene glycol methyl ether methacrylate or a mixture thereof to obtain a component 2 solution;
c) mixing the component 1 solution and the component 2 solution at a volume ratio of 10:0-8:2, wherein:
a) is to prepare a component 1 solution by using a 0.1-0.5% LAP solution to prepare a 10-30% (w/v) gelatin methacryloyl or chondroitin sulfate methacrylate solution;
b) is to prepare a component 2 solution by preparing a 0.001 g/ml-0.5 g/ml polyethylene glycol diacrylate or polyethylene glycol methyl ether methacrylate; and
in c), the component 1 solution and the component 2 solution are mixed at a volume ratio of 9:1-8:2.

14. (canceled)

15. The preparation method according to claim 7, wherein the cooling method is to place the decellularized cornea in a 4° C. environment for 15-30 minutes.

16-18. (canceled)

19. A decellularized cornea prepared by a method for preparing a decellularized cornea comprising: immersing a decellularized cornea in a monomer polymer solution, taking out the decellularized cornea, and then activating the polymerization reaction of the monomer polymer inside the decellularized cornea wherein the interior of the decellularized cornea contains and/or the surface is attached with a solidified monomer polymer solution; after being applied to a recipient cornea, the monomer polymer solution liquefies, and partially exudes from the decellularized cornea and penetrates into the space between the recipient cornea and the decellularized cornea, as well as into the recipient cornea; upon irradiation with visible light or ultraviolet light, the monomer polymer undergoes a polymerization reaction to form an adhesion layer between the decellularized cornea and the recipient cornea, wherein:
the condition for maintaining the solidified monomer polymer solution is 4-25° C.; the liquefaction condition is that the surface temperature of the recipient cornea is greater than 25° C.;
the wavelength range of the visible light is 300-800 nm, the light energy is 100-500 mW/cm², and the light irradiation time is 30 seconds to 2 minutes.

20-21. (canceled)
